(19) United States
(12) Patent Application Publication
(10) Pub. No.: US 2019/0062173 A1
(43) Pub. Date: Feb. 28, 2019

(54) PROCESS FOR MAKING SOLID PARTICLES

(71) Applicant: The Procter & Gamble Company, Cincinnati, OH (US)

(72) Inventor: Geoffrey Marc Wise, Reading, OH (US)

(21) Appl. No.: 15/901,278

(22) Filed: Feb. 21, 2018

Related U.S. Application Data
(60) Provisional application No. 62/548,524, filed on Aug. 22, 2017.

(51) Int. Cl. C01D 5/00; C08B 15/06; C07C 29/78; C08F 126/04; B01D 9/00

(52) U.S. Cl. CPC C01D 5/00; C08B 15/06; C07C 29/78; B01D 2009/0095; B01D 9/005; C08F 2810/40; C08F 126/04

(57) ABSTRACT

The invention relates to a process of creating particles of controlled size by creating them in the interstitial regions in a batch, semi-continuous, or continuous liquid phase. The method comprises making solid particles comprising: adding a precursor material to a liquid carrier to form a liquid continuous phase, wherein the concentration of the precursor material is from about 5% to about 99% by weight of the continuous liquid phase; adding an inert phase into the liquid continuous phase of step a, resulting in an inert phase and continuous liquid phase mixture having a volume fraction of the inert phase of from about 30% to about 98% and inert phase domain size of about 0.2 to about 200 μm; transforming the precursor material physically or chemically, resulting in the formation of solid particles.

### FIELD OF THE INVENTION

The invention relates to a process of forming solid particles of a controlled size by utilizing a batch, semi-continuous, or continuous process.

### BACKGROUND OF THE INVENTION

The present invention relates to a process for controlling the particle size of crystals and precipitates, particularly those that occur rapidly from a supersaturation condition. The types of compounds that may be crystallized or precipitated include pharmaceutical compounds, particles of use to the personal care industry, as well as any other compounds in industry. They may be inorganic materials, such as soluble salts of calcium, copper, magnesium, zinc or other multivalent metals, or they may be organic material, by which we mean materials that have a high concentration by weight of carbon, such as about 10% to about 95%.

Common methods for crystallization of materials useful in the pharmaceutical industry include precipitation from solution via cooling or addition of a precipitating agent, such as an anti-solvent, as well as combination of two soluble components to form an insoluble complex. These processes can be batch, semi-continuous, or continuous processes.

Crystals or particles of a target particle size are often desired due to their short dissolution times, high bioavailability, and minimal impact on product texture and appearance, as well as for compliance with safety and regulatory restrictions. Therefore, the desired particle size typically falls between about 0.1 micrometers (μm) and about 100 μm, alternatively from about 0.15 to about 10 μm.

Crystallization is inherently a difficult process to control and scale up, due to the complicated and often rapid processes of nucleation, growth and agglomeration that can be quite sensitive to formulation and process variables. Some existing processes for controlling particle size use high energy, such as ultrasonics, cavitation, milling, impinging jet, etc. in an attempt to limit the size of the formed particles. Another existing practice, particularly in the production of polymers, is emulsifying at least one of the reactants as insoluble droplets inside a second, continuous phase prior to the crystallization or precipitation event.

There also exist industrial processes for making solid particles out of the internal phase of emulsions. If the chemistry and processing conditions are carefully controlled, then the size of the emulsion droplets and concentration of reactants in the emulsion can be used to control the resulting particle size.

All of the aforementioned processes have drawbacks. In the case of the high-energy processes, there is no specific control of the particle size, only a general correlation of smaller sizes with increased energy, which may need to be very high, in contrast to a low energy density input such as less than 0.1 kJ per kg of formed material, for inexpensive industrial production. Additionally, there can be an objectionably high level of fine particles below the target size. The emulsification processes frequently use specialized chemistry and processing conditions, and have the additional difficulties of controlling mass transfer to an internal phase, and the need to remove the continuous phase in a later processing step. Frequently, the process is conducted at a lower concentration than otherwise desired, due to aggregation of the formed particles, resulting in costly and difficult purification processes.

Forming solid particles out of the internal phase of an emulsion has unique difficulties associated with mass transfer of desired and undesired materials between the emulsified droplets and the continuous phase. For example, in emulsion polymerization, the size of the formed particles can be limited at high production rates by the diffusion of monomer and initiator from the inert continuous phase to the reaction in the internal phase. Additionally, at appreciable concentrations of reagents in the droplets used for cost-effective particle-forming processes, the dramatic change in composition of the droplets as the particle-formation reaction leads to instability that is difficult to manage without sophisticated and expensive chemistry.

There remains the unmet need for an inexpensive, simple, broadly-applicable industrially feasible crystallization process for making particles and crystals having a maximum dimension of from about 0.1 to about 200 μm size range, alternatively from about 0.2 to about 10 μm.

The process disclosed herein is also useful for creating non-crystalline solid particles of a controlled size. A non-limiting example is a coacervate which is formed out of an aqueous solution containing an ionic surfactant and an ionic polymer of opposite charge, such as an anionic surfactant and a cationic polymer. The cationic polymer may be synthetic or naturally derived, such as a cationic guar or cellulose. Of particular interest are cationic polymers with charge densities greater than 1.0 meq/gram due to their coacervation properties. Such a combination of anionic surfactant and cationic polymer can be selected to precipitate out of a personal care composition upon addition of water, as in a rinsing operation, leading to deposition of the coacervate onto a desired surface, such as skin or hair, to provide a personal care benefit.

In addition to the benefits of depositing the coacervate itself, coacervate systems are often used to deposit other benefit agents, such as hydrophobic oils and anti-microbial particles, by trapping them during the coacervation process. However, this trapping process is relatively inefficient. Moreover, there is difficulty in optimizing the overall formula to simultaneously achieve the desired dilution factor that triggers the coacervation, the rheology of the formed coacervate, and the coacervate particle size, all of which are critical to the deposition efficiency in use. Below, we describe a way to use our invention to independently control the size of the coacervate particles for enhanced deposition.

### SUMMARY OF THE INVENTION

The invention relates to a method of making solid particles comprising: (a) adding a precursor material to a liquid carrier to form a liquid continuous phase, wherein the concentration of the precursor material is from about 5% to about 99% by weight of the continuous liquid phase; (b) adding an inert phase into the liquid continuous phase of step a, resulting in an inert phase and continuous liquid phase mixture having a volume fraction of the inert phase of from about 30% to about 98% and inert phase domain size of about 0.2 to about 200 μm; transforming the precursor material physically or chemically, resulting in the formation of solid particles.

### BRIEF DESCRIPTION OF THE DRAWINGS

FIG. 1 is a schematic of the multiphase system for forming the particles.

FIG. 2 Behenyl alcohol particles formed in Example 1.

FIG. 3 Behenyl alcohol particles formed in Example 2.

FIG. 4 Behenyl alcohol particles formed in Example 3.

FIG. 5 Behenyl alcohol particles formed in comparative Example 4

FIG. 6 ZPT+polyquarternium-6 particles formed in Example 5.

FIG. 7 ZPT+polyquaternium-6 particles formed in Example 6.

### DETAILED DESCRIPTION OF THE INVENTION

### A. Definitions

SOLID as used herein means a substance that has a definite volume and shape and resists forces that tend to alter its volume or shape.

PRECIPITATION as used herein means a process of producing solid particles having controlled particle size within a continuous liquid phase.

CRYSTALLINE as used herein means a material in which the constituent atoms are arranged in a three-dimensional lattice, periodic in three independent directions.

CRYSTALLIZATION as used herein means a process that produces a crystalline material.

SURFACTANT as used herein means a molecule with amphiphilic character, in which one part of the molecule has affinity for hydrophobic oil and another part of the molecule has affinity for water.

PERSONAL CARE PRODUCT as used herein means a consumer product applied to part of the human body for cosmetic purposes, such as cleaning or altering the appearance or feel of that part of the body.

LIQUID as used herein means a state of matter intermediate between that of crystalline substances and gases in which the substance has the capacity to flow under extremely small shear stresses and conforms to the shape of a confining vessel, but it is relatively incompressible, lacks the capacity to expand without limit and can possess a free surface.

PRECURSOR MATERIAL as used herein means a material that can be converted into a collection of solid particles of controlled size using a method that includes either (a) a reaction with another material or (b) a physical process.

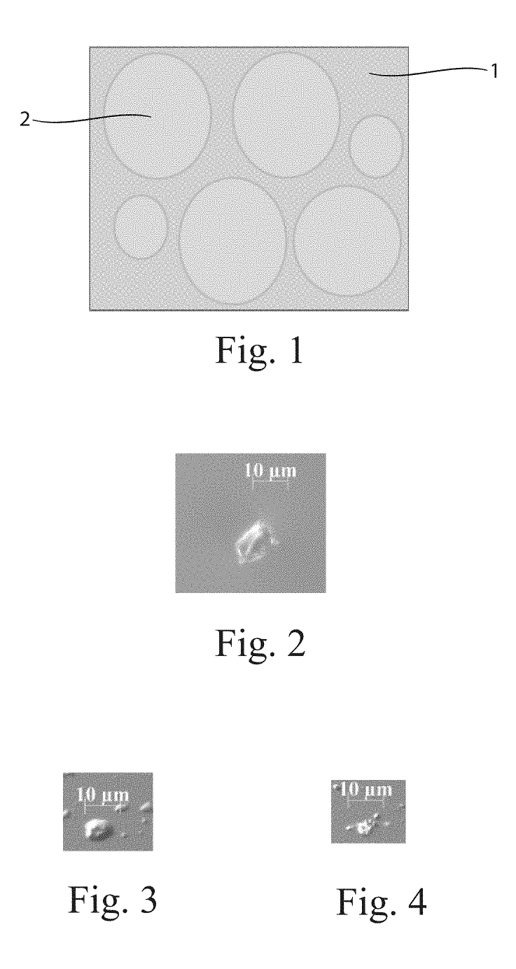

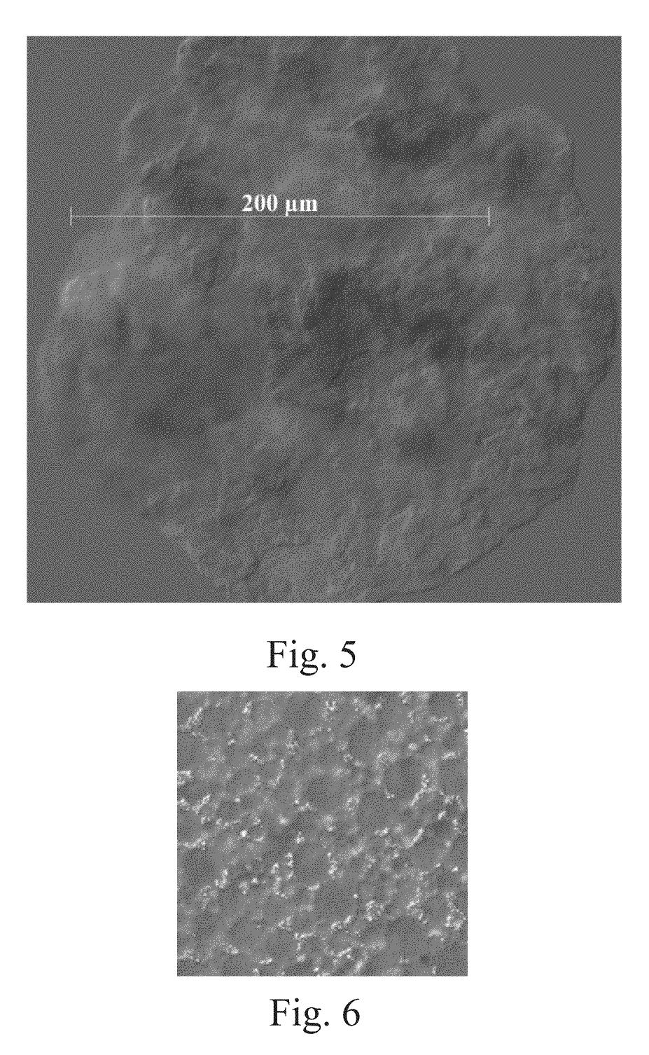

CONTINUOUS LIQUID PHASE as used herein means the combination of a precursor material and a liquid carrier. In a continuous process, the continuous liquid phase flows through a confining geometry or conduit, such as a pipe, at a specified mass flow rate that can be expressed in convenient units, such as grams per minute. In a batch process, the continuous liquid phase is present in a vessel, typically under agitation. The combination of liquid carrier and the precursor material may be a solution of a precursor material in the liquid carrier or a dispersion of the precursor material in the liquid carrier. (As shown in FIG. 1, reference point 1).

INERT PHASE as used herein means an inert liquid, an inert solid, and mixtures thereof that do not contain molecules that react chemically with the precursor material in the continuous liquid phase to form molecules of a different molecular composition. (As shown in FIG. 1, reference point 2).

LIQUID CARRIER as used herein means a liquid material or a combination of liquid materials that serves as the medium in which the formation of the desired solid particles takes place.

CONTINUOUS PROCESS as used herein means a process in which the raw materials are delivered continuously into a physical volume of constant dimensions, and the resulting product is continuously removed from this volume. The chemical and/or physical process toward the preparation of the solid of controlled particle size takes place inside said physical volume.

BATCH PROCESS as used herein means a process in which the raw materials are mixed together inside a vessel and the resulting product mixture is removed from this vessel after a desired time. The chemical and/or physical process toward the preparation of the solid of controlled particle size takes place inside said vessel.

SEMI-CONTINUOUS PROCESS as used herein means a process in which one or more sets of material combination is prepared via a batch process and the one or more sets of material combinations are then delivered continuously into a physical volume of constant dimensions, and the resulting product is continuously removed from this volume. The chemical and/or physical process toward the preparation of the solid of controlled particle size takes place inside said physical volume.

PARTICLES as used herein means distinct pieces of solid matter that substantially retain their shape and size when dispersed in a liquid carrier. The particles formed can be organic material having from about 10% to about 95% by weight carbon

### B. Description of the Method

The present invention overcomes the previous difficulties by using a high volume fraction of an inert phase controlling the size of the particles produced in the liquid continuous phase that surrounds the inert phase, as illustrated schematically in FIG. 1. By controlling the size and volume fraction of the inert phase, we can effectively create semi-enclosed three-dimensional regions or “micro-compartments” that restrict the growth of the desired solid particles beyond the volume contained in each micro-compartment. The use of an inert phase, such as a solid or a viscous liquid, avoids many of the difficulties associated with controlling the size of the desired particle. It should also be clarified that this process is different from traditional emulsification-based processes for forming solid particles which form the solid particle in the internal phase of the emulsion. By forming the particles in the liquid continuous phase, as described herein, the reaction product is much more easily collected out of the bulk, and the inert phase can be recycled back into the process. The details of this invention will be described below, wherein the term “precipitation” will be used generically to include precipitation, crystallization, and other particle-forming processes taking place in the liquid continuous phase of the multiphase composition.

1. Selection of the Liquid Carrier

The liquid carrier is the medium for carrying out the formation of the desired solid particles in the presence of the inert phase. For convenience, the liquid carrier typically has a low viscosity, from about 0.1 to about one million centipoise (cP), alternatively from about 0.2 to about ten thousand cP, alternatively from about 0.2 to about 1000 cP, wherein the viscosity is measured at the temperature of the solid-forming reaction and a shear rate of 1/sec, using any of the common viscometry methods known in the art, such as Brookfield RV cone-and-plate viscometer. Higher viscosities translate to more difficult blending and therefore more difficulty in executing the solid-forming reaction and any subsequent separation steps. The liquid carrier may be aqueous or oil-based. The combination of the liquid carrier and the precursor material forms the continuous liquid phase. Although the continuous liquid phase can often be a homogeneous fluid, it may contain additional particles or droplets of a size smaller than the inert phase, such that the solid-forming reaction encapsulates or adheres to these small entities in the pockets of space between the inert phase particles or droplets. These smaller dispersed particles or droplets may be of the same or different composition as the inert phase.

As said above, the continuous liquid phase also contain precursors or reagents that are used in the formation of the desired particles. The reagents may be present in the continuous liquid phase prior to the introduction of the inert phase, or may be added after the inert phase is dispersed. The reagents used in the solid-forming reaction may be dispersed materials rather than dissolved in the liquid carrier. For the purposes of definition, these components will be considered part of the continuous liquid phase, in contrast to the separate inert phase, described below, that is used for particle-size control.

2. Selection of the Inert Phase

The inert phase can be either a solid or a liquid, but it usually has at least the following three characteristics to be useful in an industrial process:

The inert phase should be sufficiently rigid so as to relatively retain its shape under typical processing conditions.

The inert phase should be essentially immiscible with the continuous liquid phase. Two phases A and B are essentially immiscible if a composition obtained by combining A and B results in at least two distinct phases, with one phase from about 99% to about 100% A and the other phase from about 99% to about 100% B. Essentially immiscible means less than one part per hundred of the inert phase is soluble in the continuous phase.

The inert phase can have a density substantially different from the liquid phase, so as to ease the separation after the precipitation reaction.

As used herein rigid means a particle or droplet that can withstand local shear stresses of the order of greater than about 1 Pa, alternatively greater than about 10 Pa, alternatively greater than about 100 Pa, alternatively from about 1 Pa to about 500 Pa or greater for a considerable time, such as about ten seconds while retaining its shape after the cessation of the shear stress. A rigid particle is deformed between from about 0 and about 5% from its original shape after ten seconds of application of a shear stress of from about 0 to about 1 Pa, alternatively from about 0 to about 10 Pa, alternatively from about 0 to about 500 Pa. In contrast, a non-rigid particle would deform irreversibly into fragments under the action of the shear stress within a reasonable time, such as about ten seconds. Having a substantially different density, means at least 10 kilograms per cubic meter difference in density relative to the continuous liquid phase, alternatively greater than 30 kg per cubic meter, alternatively from about 10 to about 3000 kg per cubic meter, and alternatively from about 30 to about 2500 kg per cubic meter. The inert phase may be either lighter or denser than the continuous liquid phase.

When a solid inert phase is used, it may be used as-received from a supplier in a slurry or dry powder form, or ground to a smaller size. The morphology of the micro-compartments of continuous liquid phase may be influenced by the size, shape, and volume fraction of the solid phase dispersed in the continuous liquid phase prior to the precipitation reaction, as these geometric properties of the solid phase will control the size of the micro-compartments of continuous liquid phase in which the desired particle-forming reaction is to take place. Additionally, an inert phase that is effective in bringing about the desired morphology of the formed solid particles is selected. Spherical and substantially aspherical (platelets, needles, fibers, box-like, irregular) shapes of the solid inert phase are suitable in forming the desired micro-compartment topology. The solid inert phase may be derived from biological, mineral, or synthetic sources. For solid inert phase particles with near or below one micrometer in characteristic length, a surfactant or a dispersant may be added to mitigate attractions between the small moieties of the solid inert phase. This dispersant or surfactant may be the same or a different one from that used to stabilize the particles formed as a result of the desired precipitation reaction.

When a liquid inert phase is used, it will typically be received as a liquid, then emulsified to droplets of the desired droplet size, again selecting this droplet size based on considerations of how the size of the continuous liquid phase micro-compartments relate geometrically to the phase volume and particle size of the dispersed droplets. Alternatively, the liquid inert phase may have been pre-emulsified to the desired droplet size prior to its introduction into the particle-forming process. Any of the common means of emulsifying one liquid in another can be used, including techniques employing stirred tanks, impeller, static mixers, rotor-stator mills and homogenizers; for additional common techniques and surfactants employed therein one may refer to Encyclopedic Handbook of Emulsion Technology (CRC Press).

In order for the droplets to have sufficient stability to maintain their size during the formation of the solid particles, they will typically be more viscous than the continuous phase. Academic studies of droplet breakup (e.g. “Droplet breakup in concentrated emulsions” by K. Jansen et al. starting on p. 227 on volume 45 of the Journal of Rheology, published in 2001, and the references cited therein) have established that droplets with viscosities greater than four times the viscosity of the complete emulsion are completely stable against breakup in a laminar shear flow. Most flows of industrial importance have some extensional flow character in addition to shear flow, which can induce break-up of even viscous droplets at high shear stresses, but it is known that more viscous droplets are more stable than droplets with viscosities comparable to the continuous liquid phase viscosity, and that smaller droplets are more stable than larger droplets, through a balance of hydrodynamic and interfacial forces expressed in terms of a Capillary number for laminar flow or a Weber number for turbulent flow. Typically, the droplets will be formed in a high-energy process prior to the precipitation reaction, and then a milder flow condition will be employed for the precipitation reaction. Alternatively, the inert liquid phase is emulsified to the desired droplet size and volume fraction as an integral step of the precipitation reaction.

High-viscosity (from about 1000 to about 1,000,000 cP) silicones, such as dimethicone and dimethiconol of high molecular weight, are often ideal choices as a liquid inert phase, due to their low cost, chemical inertness, low solubility in water and most organic oils, and the relative ease of processing them into high-internal-phase emulsions, as exemplified by U.S. Pat. No. 7,638,119, WO2012/031903, and references therein. Other molten or dissolved high-molecular-weight polymers are also contemplated for their use as inert droplet phases for the precipitation reaction.

A means of obtaining a narrow droplet-size distribution is the use of a high internal phase (HIP) emulsion, where the volume fraction of droplets is so high that the droplets are deformed from their spherical shape. For a unimodal droplet distribution, an emulsion will be a HIP above about 74% volume fraction of internal phase. Since perfectly unimodal distributions are difficult to achieve, an emulsion is typically a true HIP above a volume fraction of about 80%. HIPs with volume fractions below about 95%, alternatively about 90% are suitable as higher HIP volume fractions lead to excessively shear-thinning (pasty) rheological behavior, making them more difficult to process. In addition to their stability against droplet creaming or sedimenting, HIPs have additional value for the compositions in that their Plateau borders provide quite restricted micro-compartments for the precipitation reaction to be localized, as will be exemplified in some of the later examples.

3. Preparation of the Multiphase System

After selection of the liquid carrier in which the precipitation is to take place, and selection of the inert phase to provide the necessary steric hindrance, the next step is to combine them to form the desired two-phase system. Any of the common solid-liquid or liquid-liquid dispersing techniques may be used for this purpose, including those referenced in Handbook of Industrial Mixing (John Wiley and Sons).

As previously mentioned, the morphology of the micro-compartments of continuous liquid phase for the ensuing precipitation reaction is dictated by the morphology and volume fraction of the inert phase. For most applications, the volume fraction of the inert phase can be from about 30% to about 98%, alternatively from about 50% to about 95%, and alternatively from about 60% to about 90%. Lower volume fractions may provide some steric hindrance, but will not be as effective in creating narrow regions of continuous liquid phase that are helpful in limiting the size of the formed solid particles. The upper limit to the inert-phase volume fraction is curtailed by practical limitations. For a solid inert phase, the rheology of the combined system will become prohibitively viscous as the system approaches a geometry-dependent maximum packing (see Krieger & Dougherty, J. Rheology 3(1), pp. 137-152 (1959) for a detailed explanation). This maximum packing is typically in the range of 40-80%, depending on the prevailing geometry of the dispersed phase and the breadth of the distribution of sizes of inert solid particles.

For a solid inert phase, the volume fraction of inert phase in the multi-phase system can be determining by measuring the volume of a known mass of continuous liquid phase, then measuring the volume again after addition of a known mass of the added solid inert phase. For a liquid inert phase, it is more convenient to measure the specific volume of the two liquids, then assume that the specific volume of the inert phase is unchanged by the dispersion process, due to the two liquids' immiscibility when combined.

For a liquid inert phase, the maximum volume fraction is a little more flexible, as the droplets can deform in the HIP state. As previously mentioned, volume fractions of spheres above 74% and a unimodal droplet distribution create a “high-internal-phase” emulsion with narrow struts connecting pockets of continuous liquid phase of diameter roughly 0.1-0.4 times the diameter of the droplets. Higher volume fractions of inert phase can create thinner connecting channels between the micro-compartments, such that particles that bridge these interstitial regions will be more easily broken by an optional moderate shearing step downstream of the precipitation or crystallization step. Higher volume fractions also tend to narrow the particle-size distribution. Inert phase volumes above 90% are more difficult to process due to their higher rheology and tendency to invert the positions of the continuous liquid phase and inert phases, known as a phase inversion. Phase inversion can often be avoided by proper selection of the emulsifier or surfactant used to create the emulsion. It is generally the case that emulsifiers with much stronger affinities for the desired continuous phase than for the desired internal phase are more likely to avoid phase inversion. For example, when using water as the liquid carrier and a viscous oil as the desired inert phase, a hydrophilic emulsifier should be used. Those skilled in the art will be familiar with the concept of the HLB number to approximately describe the hydrophilicity vs. oleophilicity of various surfactants. High-HLB nonionic and ionic surfactants are suitable for formation of the HIP emulsions in aqueous continuous phases. Blends of low- and high-HLB surfactants are often used, with the ratio of low- to high-HLB tuned to give the appropriate HLB of the blend.

The characteristics of the emulsion or slurry, including volume fraction and inert phase size, can be determined using conventional techniques such as inline microscopy, conductivity, magnetic resonance imaging, laser scattering, and the like. The Krieger-Dougherty relationship, and extensions thereof, can often be used to infer morphology from the rheology of the dispersion.

The skilled practitioner will appreciate that the size of the formed crystals or particles will be related to the inert phase size, as well as the concentration of the reactants in the continuous liquid phase. For the process to be industrially relevant, the reactant concentration will be typically greater than 0.1% by weight, alternatively greater than about 2% by weight of the continuous liquid phase, alternatively from about 0.1% to about 90%. And as mentioned previously, suitable inert-phase volume fraction at the point of the particle-forming reaction or crystallization can be above about 30%, alternatively from about 30% to about 98%, and alternatively from about 50% to about 90%. Therefore, from geometric considerations, the desired inert phase particle or droplet diameter will be somewhat larger than the desired particle size of the main reaction product, particularly for a low concentration of reactant in the continuous liquid phase. In typical situations, the inert phase diameter may be from about 1.5 times and about 100 times the approximate diameter of the formed particles, or alternatively from about 0.2 to about 200 microns.

4. Reaction to Form the Desired Particles

Once the inert phase has been properly dispersed, the precipitation or crystallization process can proceed quickly via any means known in the art. An anti-solvent such as an electrolyte or alcohol can be added to precipitate out a solid previously dissolved. Alternatively, the introduction of another dissolved species causes a precipitation reaction, typically via a cationic-anionic interaction. Examples of these include the precipitates that form quickly when some metallic cations such as copper, zinc, and calcium contact certain anions such as carbonates. Other examples of precipitation reactions include coacervates or liquid crystals which can form between cationic polymers and anionic surfactants as they are mixed, or sometimes after the addition of water. Suitable cationic polymers for forming precipitates with anionic surfactants include those cationic polymers having charge densities of from about 1.0 to about 20 meq/gram, alternatively from about 2 to about 15 meq/gram. Alternatively, a change in temperature, pH, or solvent quality in the composition can induce the precipitation or crystallization of one of its components from the continuous phase.

Some mixing can occur to intimately mix the composition in order to generate the desired crystals or particles, but the presence of the inert phase will generally reduce the energy input needed to avoid undesirable agglomeration. The presence of the inert phase will also ease the break-up of any weak agglomerates that do form in spite of the steric hindrance provided by the inert phase. Any of the commonly-known mixing devices may be used for this purpose, such as high-pressure homogenizers, colloid mills, rotor-stator mills, static mixers, orifice plates, and the like. If it is desired to recycle the inert phase for use in subsequent production, the energy level of this recovery operation should be kept below that which will substantially reduce the inert-phase particle size. Alternatively, the separation process to concentrate the inert phase for recycling into the process may be effected prior to moderate shearing operations to break up weak agglomerates of the desired solid particles.

Since the emulsion or slurry is stabilized by the high concentration of the inert phase, the precipitation step may take place immediately after the dispersion of the inert phase into the continuous liquid phase, as would often be desirable in a continuous process, or may proceed at a more leisurely pace, as in a typical batch crystallization or precipitation process. Only a minimal level of mixing may be needed to prevent settling or creaming of the continuous liquid phase as the precipitation reaction proceeds.

For some applications, markedly non-spherical particles are desired for their enhanced surface area per volume, interfacial properties, and the like. It will become apparent to those skilled in the art that the shape of the particles formed in the semi-confined micro-compartments of continuous liquid phase may depart significantly from spherical, particularly at high volume fractions of continuous liquid phase and high concentrations of the reactant material. Both spherical and substantially aspherical particles are contemplated as results of the present invention.

It is additionally conceived that sequential reactions could be performed in the confined spaces, so as to make novel composite structures by adding an additional component which connects to the recently formed particles. In other words, the continued presence of the physically-stable inert second phase may enable the production of a composite structure of a controlled morphology that would otherwise be difficult to create without undue agglomeration.

In the case of a precipitation reaction, such a sequence of events could be represented by:

A+B+I→AB+I

AB+I+C→AB−C

Where I represents the inert phase, A and B are the two components which combine to form an insoluble compound in the continuous liquid phase around I, and C is the later component to be connected to the newly formed AB particle. The continued presence of the I phase limits the formation of an undesired AB-C-AB-C-AB-C-AB-C . . . agglomerate exceeding the target particle size of a single AB-C composite.

In a similar fashion, when the particle is formed from a single, solubilized reactant A, we have:

A (soluble)+I+NS→A (solid)+I+NS

A (solid)+I+C→A−C

In this case, NS refers to a non-solvent or any other change, such as cooling, which induces A to become solid.

The above reaction scheme may be particularly useful in creating composite structures involving cationic or anionic polymers, particularly those with a linear charge density greater than 1.0 meq/gm. Alternatively, the charge density is from about 1 meq/gm to about 20 meq/gm, alternatively the charge density is from about 2 meq/gm to about 15 meq/gm. These polymers tend to react rapidly with surfactants of the opposite charge, as may occur on the surface of a solid particle or droplet, or in solution of a cleaning or personal care composition. The present invention allows these cationic-anionic reactions to occur in a more controlled way due to the steric hindrance provided by the inert phase, thus enabling the production of controlled composite structures that may be useful for enhanced deposition. The enhanced deposition may be of the cationic-anionic complex alone, or of this complex combined with the inert phase.

The description of the invention may be further understood through the non-limiting examples outlined below. The first three examples illustrate the use of various solid inert phases to manipulate the particle size of an organic material as a representative pharmaceutical active, whereas comparative Example 4 illustrates the undesired large particle size that occurs with identical processing in the absence of the inert phase. Examples 5 and 6 exemplify the influence of the droplet size of a liquid inert phase on the particle size of a cationic-anionic complex formed. The final compositions in examples 5 and 6 are chemically identical, but morphologically different based on the particle size of the dispersed silicone phase, effected via different emulsification conditions. Examples 7 and 8 employ a different liquid inert phase to control the particle size of crystals of a model inorganic salt precipitated out of a batch crystallization process. The comparative example 9 illustrates an undesired, much larger particle size when crystallizing this salt in the absence of the inert phase. Example 10 illustrates a continuous process for controlling the size of a coacervate formed out of the continuous liquid phase of a concentrated emulsion.

C. Types of Particles Formed

1. Transition-Metal Salts of Suitable Size

Antimicrobial particles, such as zinc and copper salts, are generally more effective in personal care compositions at sizes from about 0.1 μm to about 10 μm, alternatively from about 0.1 μm to about 5 μm, alternatively from about 0.3 to about 10 μm, alternatively from about 0.3 μm to about 5 μm. Antimicrobial particles in this size range can have more efficient deposition, greater bioactivity, and improved consumer-noticeable attributes such as feel. There are several traditional ways of making these materials, including direct crystallization of the desired particle size and shape from a bulk solution, precipitation from the internal phase of an emulsion, and creation of large particles that are then reduced in a subsequent grinding, milling, or other particle-size reduction process. Each of these processes has undesired aspects such as restrictions to particular chemistries and equipment/processes that are expensive and difficult (inefficient).

The process described herein minimizes these difficulties by introducing a second, inert internal phase in one of the reactant streams prior to the precipitation or crystallization reaction. The presence of this phase presents a physical barrier to the formation of large particles; any larger particles formed are easily fractured due to the very thin connections between them that were formed in the narrow regions between the bubbles or droplets of the inert phase.

2—Enriched Coacervates

Manufacturers of consumer products has used high-charge-density cationic polymers, such as polyDADMAC (Polyquaternium-6; poly(diallyl dimethyl ammonium chloride)) to form liquid crystals in shampoo by mixing polyDADMAC with anionic surfactant (e.g. sodium laureth sulfate)—[e.g. US20080206355A1]. These “in-situ” coacervates function like traditional coacervates in that they deposit on surfaces of interest (skin, hair, scalp), and frequently act as deposition aids by bringing nearby particles (silicone droplets, anti-microbial actives, etc.) with them to the target surface, but unlike coacervates created by the consumer during the rinsing step, they are pre-formed by the manufacturer of the personal care composition.

It is desired to control the particle size of the in-situ coacervates to from about 0.1 to about 50 μm, alternatively from about 0.5 to about 10 μm for consumer feel benefits, maintenance of lather, etc., but has been traditionally difficult to achieve due to the strong driving force of forming the cationic-anionic complex. Variables such as the mixing energy during the contact of the cationic polymer with the anionic surface, and the composition (electrolyte, surfactant level, etc.) of the medium are helpful, but it is still difficult to maintain the particles in the desired size range without objectionable agglomeration.

Furthermore, when the coacervate particles are tasked with depositing other materials, it is desired to enrich the relative concentration of the materials to be deposited relative to the coacervate, to result in a greater deposition of the high-value materials (HVM). HVMs include, but are not limited to, conditioning agents and pharmaceutical actives, ZPT, antidandruff agents and combinations thereof. The interstitially controlled particle formation process of the present invention has additional value when the particle formation occurs in the presence of an enhanced concentration of the HVM relative to their composition in the bulk, thus enriching the concentration of the HVM in the deposited floc.

### Examples

The following examples illustrate embodiments of the invention described herein. All parts, percentages, and ratios herein are by weight unless otherwise specified. Some components may come from suppliers as dilute solutions. The amount stated reflects the weight percent of the active material.

| Example # | Particle formed | Inert Phase |
|---|---|---|
| 1 | Behenyl alcohol | Sodium chloride |
| 2 | Behenyl alcohol | Cationic guar |
| 3 | Behenyl alcohol | Basic zinc carbonate |
| Comparative 4 | Behenyl alcohol | None |
| 5 | Zinc pyrithione - cationic polymer | Dimethicone |
| | complex | |
| 6 | Zinc pyrithione - cationic polymer | Dimethicone |
| | complex | |
| 7 | Sodium sulfate | Polydecene |
| 8 | Sodium sulfate | Polydecene |
| Comparative 9 | Sodium sulfate | None |
| 10 | Cellulose - sodium laureth sulfate | Dimethicone |
| | coacervate | |

Descriptions of Raw Materials Used in the Examples:

Behenyl alcohol is “Lanette 22” from BASF Corporation.

Cellulose is Polyquaternium-10 from Union Carbide Amerchol.

Decyl glucoside used is “Plantaren 2000 N UP”, a 50% solution in water supplied by BASF.

Dimethicone is a polydimethylsiloxane from Momentive Performance Materials, with an approximate viscosity of 350,000 cP at 20° C. and an approximate density of 0.96 g/mL at 20° C.

Glycerin is supplied by Procter & Gamble Chemicals

Guar is “Jaguar C-500” from Rhodia. Microscopy images of it slurried in laureth-4 indicate irregular but primarily spherical shape, with a typical maximum diameter of 40 μm.

Laureth-4 is “Brij L4” from Croda. It is a free-flowing liquid with a density of −0.92 g/mL

Polydecene is Thorcosyn 150 from Van Horn Metz (Beaumont, Tex.) with an approximate viscosity of 2700 cP at 27 C and approximate density of 0.82 g/mL at 20° C.

Polyquaternium-6 is “Mirapol 100S”, a 6.2 meq/gram cationic polymer from Solvay. It is supplied as a 31.5% aqueous solution.

Sodium chloride is from Morton Salts. Microscopy images of it slurried in laureth-4 indicated a cubic or box-like shape, with a typical maximum diameter of 250 μm.

Sodium laureth sulfate is a 25% by weight aqueous solution from Stepan, Matamoros, Mexico.

Sodium sulfate is supplied by Sigma Aldrich.

Zinc carbonate is a basic zinc carbonate supplied from Pan-Continental Chemical Corporation. Microscopy images of it slurried in laureth-4 indicate irregular but primarily spherical shape, with a supplier-reported mean particle size of 5 μm.

Zinc pyrithione is a 49% active “fine-particle-size ZPT” from Lonza, with a mean particle size of roughly 0.2-0.5 μm, and containing a low level of a Darvan anionic surfactant.

Compositions and results for Examples 1-4:

| | Example | | | | | | | |
|---|---|---|---|---|---|---|---|---|
| | 1 | | 2 | | 3 | | Comparative 4 | |
| Laureth-4 | 36 | g | 47 | g | 53.1 | g | 82.8 | g |
| Behenyl alcohol | 4.0 | g | 5.2 | g | 5.9 | g | 9.2 | g |
| Sodium chloride | 116 | g | 0 | | 0 | | 0 | |
| Guar | 0 | | 59.5 | g | 0 | | 0 | |
| Zinc carbonate | 0 | | 0 | | 91.2 | | 0 | |
| Approximate | 100 | mL | 100 | mL | 100 | mL | 100 | mL |
| reactor volume | | | | | | | | |
| Largest crystal size | 20 | μm | 9 | μm | 5 | μm | 250 | μm |

### Procedure for Examples 1-3 and Comparative 4:

For each crystallization, a hot water bath is set up in a 2000-mL beaker equipped with a 2″ long oval-shaped magnetic stirrer at 400 rpm. The 2000-mL beaker is filled 90% with tap water, and the temperature set point of the bath is 75° C.

Laureth-4 is poured into a 4 cm, 8-ounce glass jar, and then the behenyl alcohol pellets is added. The 4 cm, 8-oz jar is then partially immersed in the water batch such that the water level is at or above the top of the organic liquid. A 3 cm diameter impeller is inserted and stirring commenced at 300-500 rpm, selected to be sufficient to agitate the jar contents for each condition.

The amount of inert solid phase, if used, is then added to the stirring organic liquid.

When the water bath temperature exceeds 74° C., the water-bath temperature is held at 74-76° C. for five minutes to ensure that all of the behenyl alcohol has melted, and then the heater is turned off.

The composition is allowed to cool ambiently (approximate room temperature 21° C.) while maintaining the water-bath stirrer and organic liquid impeller at their previously mentioned agitation speeds. This process takes approximately four hours.

Once the water bath cooled below 32° C., a 3-mL plastic pipette is used to draw 2 gram of the composition into a plastic weigh boat, where it is combined with 10 g of laureth-4. After allowing a few minutes for the inert solid phase to settle, a small amount of the diluted composition is dabbed onto a microscope glass slide, and covered with a glass coverslip. Five distinct 200 μm×200 μm areas of the prepared microscopy sample are selected at random while imaging with differential interference contrast (DIC) optics at 400× on a Zeiss Axioscope. The image with the largest observed particle size is selected for inclusion in the Figures section of this filing, and this particle size is reported in the table above.

### Example 5: ZPT-DADMAC Particles Created into a HIP Emulsion

An 80% by weight emulsion of the dimethicone in a continuous liquid phase of glycerin and decyl glucoside also containing zinc pyrithione is made by first blending 42 g of glycerin and 14 g of 50% decyl glucoside with a 2.5 inch marine impeller in a 450-mL plastic 3.5 inch diameter tub, then adding 18.75 g of 49% active fine-particle-size ZPT, stirring one minute at 150 rpm, then top-adding 300 g of the dimethicone and mixing for 20 minutes at 200 rpm to create the HIP emulsion. An amount of 200 g of this mixture is then transferred to a second 450-mL 3.5 inch diameter plastic tub equipped with 2.5 inch marine impeller, and 5 g of a 10% Polyquaternium-6 solution is added while stirring at 375 rpm to create the structure depicted in FIG. 6, imaged at 400× under cross-polar microscopy using a Zeiss Axioscope.

### Example 6

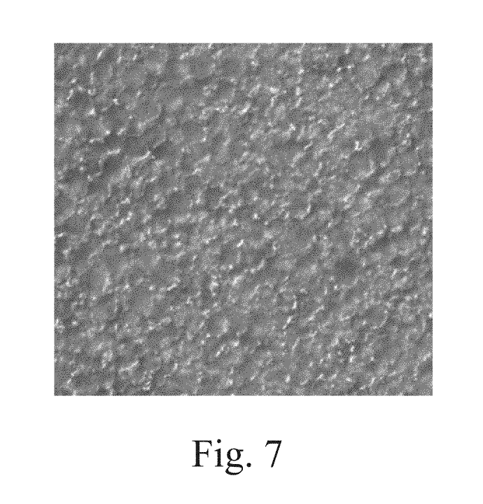

An 85% by weight emulsion of dimethicone in a continuous phase of glycerin, decyl glucoside and zinc pyrithione is made by first blending 25.5 g of glycerin and 9 g of decyl glucoside with a 2.5″ marine impeller in a 450-mL 3.5″ diameter plastic tub, then adding 18.75 g of 49% active fine-particle-size ZPT, stirring one minute at 150 rpm, then top-adding 300 g of the dimethicone and mixing for 20 minutes at 400 rpm. This is followed by addition of 16 additional grams of glycerin and 5 additional grams of the decyl glucoside to arrive at the same composition as Example 5 prior to the cationic polymer addition, but with a smaller silicone particle size due to the higher HIP volume fraction at the time of dimethicone emulsification. After an additional 5 minutes of mixing at 350 rpm, 200 g of the composition is transferred to another 450-mL plastic tub equipped with a 2.5″ marine impeller, then 5 g of a 10% polyquaternium-6 solution at 375 rpm to create the structure depicted in FIG. 7, which is imaged at 400× under cross-polar microscopy using a Zeiss Axioscope. FIGS. 6-7 are about 100 microns by 100 microns.

Examples 7, 8 and Comparative 9 illustrate the ability to control the particle size of sodium sulfate via inclusion of an inert phase of polydecene droplets, as illustrated in the following table:

| | | | | | Comparative |
|---|---|---|---|---|---|
| | Example 7 | | Example 8 | | Example 9 |
| Crystallization batch size | 100 | g | 100 | g | 120 g |
| Polydecene volume fraction | 72% | | 84% | | 0% |
| Polydecene droplet size | 10-100 | μm | 5-20 | μm | Not applicable |
| Maximum dimensions of | 150 | μm | 80 | μm | More than |
| formed sodium sulfate | | | | | 400 μm |
| crystals | | | | | |

A more detailed description of the production of these three examples follows:

### Example 7

An amount of 72 grams of SLE1S is added to 18 grams of a 20% by weight aqueous solution of sodium sulfate with impeller mixing of the 4-cm-diameter container with a 3-cm-diameter impeller, followed by addition of 210 grams of polydecene. The composition is mixed with impeller mixing of the 4-cm-diameter container with a 3-cm-diameter impeller at 300 rpm for approximately five minutes to form a 70% by weight oil-in-water emulsion. Then, 90.9 grams of this emulsion is combined with 9.1 g of sodium sulfate powder into a 200-mL glass jar and mixed at moderate agitation while immersing the glass jar in a 33° C. water bath for five minutes. The composition is rapidly cooled by replacing the water bath with an ice water bath while maintaining the moderate agitation of approximately 150 rpm. After ten minutes, the cooled composition is imaged with microscopy to determine the maximum crystal size obtained.

### Example 8

An amount of 150 g of the 70% by weight oil-in-water emulsion from Example 7 is combined with 105 g of polydecene and mixed at 600 rpm to form an 82% by weight oil-in-water emulsion. An amount of 94.8 g of this emulsion is combined with 5.22 grams of sodium sulfate powder into a 200-mL glass jar and mixed at moderate agitation while immersing the glass jar in a 33° C. water bath for five minutes. The composition is then rapidly cooled by replacing the water bath with an ice water bath. After ten minutes, the cooled composition is imaged with microscopy to determine the maximum crystal size obtained.

### Comparative Example 9

An amount of 75 g of an aqueous 20% by weight solution of sodium sulfate is combined with 20 g of SLE1S with impeller mixing in a 200-mL glass jar, followed by addition of 25 g of sodium sulfate powder. The resulting composition is mixed at moderate agitation while immersing the glass jar in a 33° C. water bath for five minutes to complete the dissolution of the added powder. The composition is then rapidly cooled by replacing the water bath with an ice water bath. After ten minutes, the cooled composition is imaged with microscopy to determine the maximum crystal size obtained.

### Example 10

Example 10 is a continuous process for forming a coacervate with size controlled by the inclusion of a continuously formed concentrated emulsion of dimethicone as the inert phase. An aqueous solution of 0.5% by weight cellulose, 0.8% by weight sodium chloride and 10% by weight active sodium laureth sulfate is created by stirring the cellulose powder into water, then combining with the sodium laureth sulfate and sodium chloride using impeller mixing. Into a stream of 40 g/min of this solution 200 g/min of dimethicone are added through the inlet of 24 elements of 10-mm SMX (Sulzer, Switzerland) static mixer to form a concentrated emulsion of the dimethicone in the surfactant. The outlet of the static mixer is connected via a 13-mm diameter pipe to a second set of 18 elements of 15-mm SMX static mixers to blend in an additional 30 g/min water stream to create a coacervate from the cellulose polymer. A liter of the resulting composition is collected and allowed to separate by gravity, to enable the silicone droplets to rise to the top of the vessel. The coacervate particles are collected and the particle size is ascertained by Focused-Beam Reflectance Measurement (Mettler-Toledo, Columbus Ohio). The resulting particle size is compared to the coacervate particle size from the same process, but with the dimethicone flow rate reduced to 100 g/min, and then a third sample is produced with the dimethicone pump turned off.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making solid particles comprising:
a) adding a precursor material to a liquid carrier to form a continuous liquid phase, wherein the concentration of precursor material is from about 5% to about 99% by weight of the continuous liquid phase;
b) adding an inert phase into the continuous liquid phase of step a, resulting in an inert phase and the continuous liquid phase mixture having a volume fraction of the inert phase of from about 30% to about 98% and an inert phase domain size of about 0.2 to about 200 μm;
c) transforming the precursor material physically or chemically, resulting in the formation of solid particles.

2. The method of claim 1, wherein the inert phase comprises a liquid with a viscosity of from about 100 times to about 1,000,000 times the viscosity of liquid stream, wherein the viscosity is measured at a shear stress of 10 Pa and at the temperature corresponding to the temperature at the initiation of the formation of solid particles.

4. The method of claim 1, wherein the density of the inert phase is from about 10 kg per cubic meter to about 3000 kg per cubic meter different than the density of the continuous liquid phase.

5. The method of claim 1, wherein the physical transformation is initiated by a change selected from the group consisting of temperature, pressure, an addition of a liquid, an addition of a solid, an addition of a salt, an evaporation of a portion of the continuous liquid phase, and combinations thereof.

6. The method of claim 1, wherein step c involves a chemical change between the precursor material and a reagent added as a component into the mixture of the inert phase in the continuous liquid phase.

7. The method of claim 6, wherein the reagent is added into the mixture of the inert phase in the continuous liquid phase as a neat liquid or in powder form.

8. The method of claim 1, wherein the continuous liquid phase comprises a cationic or anionic polymer with a charge density from about 1 meq/gram to about 20 meq/gram.

9. The method of claim 1, wherein the continuous liquid phase comprises a cationic or anionic polymer with a charge density from about 2 meq/gram to about 15 meq/gram.

10. The method of claim 1, wherein the continuous liquid phase contains from about 5% to about 90% by weight of a soluble salt of calcium, copper, magnesium, or zinc

11. The method of claim 1, wherein the formed particles have a maximum dimension of from about 0.1 to about 200 μm.

12. The method of claim 11, wherein the formed particles have a maximum dimension of from about 0.2 to about 10 μm.

13. The method of claim 1, wherein the particles formed are organic material having from about 10% to about 95% by weight carbon.

14. The method of claim 1, wherein the particle-forming process uses a mixing implement selected from the group consisting of impeller, static mixer, rotor-stator mixer or combination thereof.

15. The method of claim 1, wherein the total energy inputted to step c is from about 0 to about 0.1 kJ per kg of solid particle formed.

16. The method of claim 1, wherein the total energy inputted to step c is from about 0.0001 kJ/kg to about 0.1 kJ per kg of solid particle formed.

17. The method of claim 2, wherein the inert phase comprises from about 50 weight % to about 95 weight % of the volume of the mixture of the inert phase and the continuous liquid phase at the start of step c.

18. The method of claim 2, wherein inert phase comprises a silicone oil with a viscosity of from about 1000 to about 1,000,000 cP.

19. The method of claim 1, wherein inert phase is separated from the formed solid particles after completion of step c for recycling of inert phase back into step a.
